(19) United States
(12) Patent Application Publication
(10) Pub. No.: US 2025/0051717 A1
(43) Pub. Date: Feb. 13, 2025

(54) PROCESS FOR PRODUCING DRIED LACTOBACILLUS CELLS

(71) Applicant: Novozymes A/S, Bagsvaerd (DK)

(72) Inventors: Michael SCHILLING, Lyngby (DK); Tobias THIELE, Berlin (DE); Magdalena UHL, Berlin (DE)

(73) Assignee: Novozymes A/S, Bagsvaerd (DK)

(21) Appl. No.: 18/723,004

(22) PCT Filed: Dec. 20, 2022
(86) PCT No.: PCT/EP2022/086859

§ 371 Date: Jun. 21, 2024

(30) Foreign Application Priority Data
Dec. 23, 2021 (WO) PCT/IB2021/062249

(51) Int. Cl. C12N 1/20; C12N 1/04; C12R 1/25

(52) U.S. Cl. CPC C12N 1/205; C12N 1/04; C12R 2001/25

(57) ABSTRACT

The present disclosure provides a process for producing dried Lactobacillus cells. In one aspect, the process leads to increase in the heavy metal binding capability of Lactobacillus cells. In one aspect, a process for producing dried Lactobacillus cells comprises fermenting Lactobacillus cells in a fermentation medium. A fermentation product comprising the Lactobacillus cells is obtained after fermenting the Lactobacillus cells. The fermentation product is adjusted to a pH range between pH 8 and 11. The fermentation product is optionally concentrated before or after adjusting to the pH range between 8 and 11. The pH adjusted fermentation product is thereafter dried.

### REFERENCE TO A DEPOSIT OF BIOLOGICAL MATERIAL

This application contains a reference to a deposit of biological material, which deposit is incorpo-rated herein by reference. For complete information see last paragraph of the description.

### FIELD OF THE INVENTION

The present invention relates to a process for producing dried Lactobacillus cells. The present invention particularly relates to a process for producing dried Lactobacillus cells that remove heavy metal cations.

### BACKGROUND

Heavy metals such as lead, cadmium, arsenic, etc are harmful to human health as they accumulate in the body. Heavy metals have negative effect on nearly all organs of a human body. Heavy metal poisoning is a common human health condition in some developing countries despite of recent improvements.

Lead poisoning, and more generally lead exposure, can cause irreversible damage to children. Lead is known as environmental pollutant that exerts neurotoxic effects on human health. High exposure to lead can cause seriously damage to the kidney, liver, central nervous and hematologic systems. While the impact of lead on the system appears relatively dose-related, the US Centers for Disease Control (CDC) reported that there was no safe level of exposure to lead. There are negative health effects of lead even after low dose exposure. Blood lead concentration is the most commonly used measure of lead exposure, although it represents only about 1% of the total body burden of lead, the remainder being in soft tissues and bones. WHO recommends blood lead levels less that 5 g/dL, but levels below 5 g/dL of lead is also harmful for children's cognitive development which in turn affects the intelligence quotient (IQ) of children (World Health Organization, WHO Guideline for the clinical management of exposure to lead, 2021; Lanphear, B. P., et al., Low-level environmental lead exposure and children's intellectual function: an international pooled analysis. Environ Health Perspect, 2005, 113(7): p. 894-9). Heavy metals are accumulated in plants and animals, and eventually accumulate in human beings after being ingested with food. In some developing countries, 80% of daily lead intake is primarily from food which is approximately 12 g/day.

There are many microbes which have heavy metal binding properties. Some of the microbes having heavy metal binding properties are used to remove heavy metal from the human body. WO2014032375 titled, “Strain of Cadmium-removing Lactobacillus plantarum bacterium, and uses of the same” relates to a strain which can be used as an active ingredient to remove cadmium that is accumulated in human body.

The microbes which have heavy metal binding properties need to be stable when consumed as therapeutics or probiotics. There are various processes to prepare therapeutics or probiotics of the microbes. JP2020022392 A2 titled, “METHOD FOR PRODUCING FREEZE-DRIED LACTIC ACID BACTERIA CELLS” relates to a method for producing freeze-dried lactic acid bacteria cells by dispersing the lactic acid bacteria cells into a dispersion medium and freeze dried after adjusting the pH of the dispersion medium. But the heavy metal binding capability may vary for different strains based on the stability of the microbes. In addition, many microbes may not retain the heavy metal binding capacity after going through the manufacturing process steps. There is a need to retain or increase the binding capacity of the microbes to heavy metals post manufacturing process of such microbes.

### SUMMARY OF THE CLAIMED INVENTION

The present invention provides a process for producing dried Lactobacillus cells. In one aspect, the process leads to increase in the heavy metal binding capability of Lactobacillus cells.

In one aspect, a process for producing dried Lactobacillus cells comprises fermenting Lactobacillus cells in a fermentation medium. A fermentation product comprising the Lactobacillus cells is obtained after fermenting the Lactobacillus cells. The fermentation product is adjusted to a pH range between pH 8 and 11. The fermentation product is optionally concentrated before or after adjusting to the pH range between 8 and 11. The pH adjusted fermentation product is thereafter dried.

The Lactobacillus cells bind to heavy metal cations in vitro and/or in vivo. The in vitro binding of the heavy metal cations to dried Lactobacillus cells may be detected by incubating the dried Lactobacillus cells with a medium containing heavy metal cations. The incubated Lactobacillus cells are centrifuged to separate the Lactobacillus cells and heavy metal cations. Thereafter, the supernatant is collected to measure the heavy metal cations concentration in the supernatant. The in vivo binding of the heavy metal cations to dried Lactobacillus cells is detected by measuring the reduction of the heavy metal cations in blood and organs.

### BRIEF DESCRIPTION OF THE DRAWINGS

A more complete understanding of the present invention may be derived by referring to the detailed description and claims when considered in connection with the Figures.

FIG. 1 shows lead ions (Pb²⁺) in blood, brain, kidney and liver of respectively healthy male C57BL/6 mice (not challenged), non-treated male C57BL/6 mice challenged with a single oral dose of PbAc2 (disease), DSM 33464 treated male C57BL/6 mice challenged with a single oral dose of PbAc2 and Dimercaptosuccinic acid (DMSA) treated male C57BL/6 mice challenged with a single oral dose of PbAc2. Median values of 5 animals are shown.

FIG. 2 shows qPCR analysis of tight junction proteins in the small intestine measured as the expression levels of the tight junction proteins occluding, claudin-1, zonulin-1 (ZO-1) and zonulin-2 (ZO-2) in male C57BL/6 mice challenged with a single oral dose of PbAc2. Compared was healthy mice (not challenged), non-treated challenged mice (disease), DSM 33464 treated challenged mice and DMSA treated challenged mice. Median values of 5 animals are shown.

FIG. 3 shows Pb²⁺ adsorption of freeze-dried Lactobacillus cells derived from three different fermentation-(down-stream) processes: HH10F39D02: Freeze dried cells without pH adjustment before freeze drying (reference), HH10F39D04: Freeze dried cells with pH adjustment to pH9 before freeze drying, and HH10F39D05: Freeze dried cells with pH adjustment to pH10 before freeze drying. The Pb²⁺ adsorption is shown as relative percentage where HH10F39D02 is used as reference for the other cells and is set to 100%.

FIG. 4 shows a graph of flow cytometric determination of cell viability of freeze-dried Lactobacillus cells derived from three different fermentation-(down-stream) processes: HH10F39D02: Freeze dried cells without pH adjustment before freeze drying (reference), HH10F39D04: Freeze dried cells with pH adjustment to pH9 before freeze drying, and HH10F39D05: Freeze dried cells with pH adjustment to pH10 before freeze drying.

FIG. 5 shows high resolution microscopy of freeze-dried Lactobacillus cells with and without Pb+. First row: Pb²⁺ and HH10F39D02 (freeze dried cells without pH adjustment before freeze drying), second row: Pb²⁺ and HH10F39D04 (freeze dried cells with pH adjustment to pH9 before freeze drying), and third row: Freeze-dried Lactobacillus cells with no Pb²⁺ added.

### DEFINITIONS

The disclosed embodiments relate to processes for producing dried Lactobacillus cells.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. For the sake of brevity and/or clarity, well-known functions or constructions may not be described in detail.

As used herein, the singular forms “a,” “an,” and “the” are intended to include the plural forms as well, unless the context clearly indicates otherwise.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

The term “consisting of” means including, and limited to, whatever follows the phrase “consisting of.” Thus, the phrase “consisting of” indicates that the listed elements are required or mandatory, and that no other elements may be present. The term “consisting essentially of” means including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, “heavy metal” refers to a metallic chemical element that has a relatively high density and is toxic or poisonous at low concentrations and include without limitations lead, cadmium, arsenic and mercury.

As used herein, “lead binding product” refers to a product that binds to lead ions e.g. in the gastrointestinal (GI) tract of the human body. Lead binding in the GI tract may e.g. be measured in vivo as the reduction of lead in a blood sample obtained from a person after consumption of a lead binding product compared to a blood sample from the same person without consumption of the lead binding product, or by measuring lead ions excreted in the human faeces of a person before and after receiving the lead binding product.

As used herein, “cryoprotectant” refers to a substance protecting against the harmful effects of low or freezing temperatures, such as damage to cells during for example freeze-drying or freezing processes. In addition, in the case of freeze-drying or drying, a cryoprotectant confers to the dried elements some stability through the drying process. The action of the cryoprotectant will reduce loss of activity or viability during the manufacturing process and subsequently, its action improves the activity/viability of the micro-organisms during storage.

As used herein, “freeze-drying” is used interchangeably with lyophilisation, lyophilization, or cryodesiccation, and is used in its regular meaning as the cooling of a sample, resulting in the conversion of freeze-able solution into ice, crystallization of crystallisable solutes and the formation of an amorphous matrix comprising non-crystallizing solutes associated with unfrozen mixture, followed by evaporation (sublimation) of water from the amorphous matrix. In this process the evaporation (sublimation) of the frozen water in the material is usually carried out under reducing the surrounding pressure to allow the frozen water in the material to sublimate directly from the solid phase to the gas phase. Freeze-drying typically includes the steps of pretreatment, freezing, primary drying and secondary drying. The great advantage of freeze drying is to stabilize the materials for storage.

As used herein, “spray drying” is a drying method where a solution or suspension containing microbial cells is sprayed into a hot drying medium, whereby the microbial cells are dried. The mixture to be sprayed can be present in the form of a solution, an emulsion, a suspension or dispersion. The mixture is atomized into millions of individual droplets with the aid of a nozzle or a spraying wheel, drastically increasing the surface. The solvent, such as water, is immediately evaporated by the hot air and is discharged. Moreover, the microbial cells are spray-dried alone. The spray drying or atomization method can be distinguished from other drying methods since the use of a nozzle or similarly acting means is required, such as a unary nozzle, hollow cone nozzle, pressure nozzle, binary nozzle externally mixing, pneumatic nozzle, binary nozzle internally mixing, atomizing disk or ultrasonic atomizer. Spray drying methods are described in the prior art and are familiar to the person skilled in the art (see Gardiner et al., Teixeira et al. (supra) or EP74050 and EP285682). Devices are known and described as relevant, such as the mini spray dryer B-191 or B-290 by Buechi Labortechnik AG (Germany) or SD-6.3-R by GEA Niro (Denmark). It is further known that arbitrary adjuvants and additives can be used.

As used herein, “essential minerals” are chemical elements required as essential nutrients by the human body to perform functions necessary for life and are known to the person skilled in the art. Non-limiting examples of “essential minerals” include sodium, potassium, phosphorus, magnesium and calcium.

While certain aspects of the present disclosure will hereinafter be described with reference to embodiments thereof, it will be understood by those of ordinary skill in the art that various changes in form and details may be made therein without departing from the spirit and scope of the present invention as defined by the claims.

### DETAILED DESCRIPTION OF THE INVENTION

In one aspect the invention relates to a process for producing dried Lactobacillus cells. In a further aspect, the process leads to increase in the heavy metal binding capability of Lactobacillus cells. The process for producing dried Lactobacillus cells comprises fermenting Lactobacillus cells in a fermentation medium. A fermentation product comprising the Lactobacillus cells is obtained after fermenting the Lactobacillus cells. The fermentation product is adjusted to a pH range between pH 8 and 11. The fermentation product is optionally concentrated before or after adjusting to the pH range between 8 and 11. The pH adjusted fermentation product is thereafter dried.

In order to increase the heavy metal binding properties of Lactobacillus cells, the Lactobacillus cells are fermented, and the fermentation product is adjusted to the pH range between 8 and 11. The inventor surprisingly found that adjusting the fermentation product comprising Lactobacillus cells to a pH in the range between 8 and 11, preferably a pH in the range between pH 9 and 10 increases the heavy metal binding capability of Lactobacillus cells.

In an embodiment of the process, the pH adjusted fermentation product is dried using drying techniques such as freeze drying, spray drying or combination thereof.

In a preferred embodiment of the process, the pH adjusted fermentation product is dried using freeze drying technique. The freeze drying may be carried out at a temperature ranging between −60° C. and +50° C. and for a time ranging between 12 hours to 120 hours. In an embodiment the freeze drying is carried out at a temperature ranging between −45° C. and +30° C. and for a time ranging between 24 hours to 96 hours. In another embodiment the freeze drying is carried out at a temperature ranging between −30° C. and +20° C. for about 66 hours.

In an embodiment of the process, the pH adjusted fermentation product is dried using spray drying technique. The pH adjusted fermentation product is spray dried using any spray dryer known in the art of drying microbial products.

In an embodiment of the process, the binding of heavy metal cations to the dried Lactobacillus cells is higher compared to binding of heavy metal cations to dried Lactobacillus cells prepared at pH less than 8 or more than 11.

In an embodiment of the process, the binding of essential minerals by the dried Lactobacillus cells is such that the binding is not leading to deficiency of the essential minerals in the body. In an embodiment, essential minerals are not impacted by binding to the Lactobacillus cells.

In a preferred embodiment of the process, the fermentation product is centrifuged to concentrate the fermentation product before or after adjusting to the pH range between 8 and 11.

In a specific embodiment, the fermentation product or concentrated fermentation product contains one or more additives. In a further embodiment, the one or more additives is a cryorptectant and/or a stabilizer. In an embodiment, the cryoprotectant is glucose, lactose, raffinose, sucrose, trehalose, adonitol, glycerol, mannitol, methanol, polyeth-ylene glycol, propylene glycol, ribitol, alginate, bovine serum albumin, carnitine, citrate, cysteine, dextran, dimethyl sulphoxide, sodium glutamate, glycine betaine, glycogen, hypotaurine, peptone, polyvinyl pyrrolidone, or taurine, mammalian milk oligosaccharides, chitin, chitosan, casein, yeast, yeast extract, single cell protein, mycoproteins, other disaccharides or polysaccharides, or mixtures thereof. In a preferred embodiment, the cryprotectant is a dextrin such as Nutriose FM06.

### Lactobacillus Cells

The Lactobacillus cells suitable for the process of present invention bind heavy metals.

In an embodiment of the process, the Lactobacillus cells are Lactobacillus plantarum cells. Lactobacillus plantarum is also called Lactiplantibacillus plantarum. In one embodiment, the Lactobacillus plantarum is Lactobacillus plantarum as deposited at the Leibniz Institute DSMZ—German Collection of Microorganism and Cell Cultures with accession number DSM 33464. Lactobacillus plantarum as deposited with accession number DSM 33464 is sold under the trademark Smartguard™.

Data has shown good lead (Pb) tolerance of Lactobacillus plantarum DSM 33464 when cultured in vitro in a medium containing lead. The data has shown that this strain is able to bind lead in vitro under physiologically relevant pH and temperatures. Lead binding with this strain occurs over a time range that is considered relevant for GI passage time, and to a degree (10¹¹ CFU binds 25 mg Pb) in which there is reason to believe that a daily dose of 10⁹ CFU of this strain will bind and thus render a significant part of the daily expected ingested lead in humans. Also, L. plantarum strain DSM 33464 has undergone gastric and intestinal survival assays which furthermore have been correlated to lead binding in order to demonstrate binding of lead to this strain throughout the GI tract passage. The survival was assessed in the absence of any additional ingredients (“fasted” state), in the presence of 1:1 milk containing 3.8% fat (“fed” state), in the presence of a Yingkangwei Multivitamin supplement (“fasted/vit” state), and in the presence of both Yingkangwei Multivitamin supplement and milk 3.8% fat (“fed/vit” state). Viability of the cells was evaluated by plate counts on MRS agar (37 C, 48 h, anaerobic incubation) at time TO, 10 min (oral phase), 120 min (gastric phase), 240 min (small intestinal phase). Percentage of survival was calculated as referred to T0. Results indicated that viability was well maintained for the strain in the oral and gastric phase, with a maximum of a 0.5 log decreased after 120 min co-incubation in all tested conditions. In fed-state, up to 10⁸-10⁹ CFU were still obtained after 240 min co-incubation. In fasted state, lower number of cells were measured, especially in the presence of a supplementary vitamin supplement but still reaching 10⁵ CFU/mL at the end of the assay. The results showed that lead is bound to the Lactobacillus cell surface and would prevent the uptake over the gastro-intestinal (GI) system, distribution via blood stream and harmful interaction with relevant proteins and cell tissues. The lead binding efficacy of this strain was demonstrated in three animal models (not published). At first, the L. plantarum strain was applied in a mice model of chronic exposure, in which the mice were dosed with very high lead doses, and treatment versus prevention with this strain was investigated. As comparator, dimercaptosuccinic acid (DMSA) representing the chelation therapy was used. In the third model further described in example 1, the reduction of blood lead level was further investigated in an acute mouse model to investigate the lead uptake under more relevant conditions such as moderate lead doses and without inducing organ damage. In all mouse models, the supplementation of this strain was able to impart significant lowering of blood lead levels. In most models, a significant decrease of the lead content in brain, liver, and kidney tissue of the mice could be demonstrated with this strain in comparison with the control group. The blocking of lead uptake via the GI tract is further supported by the anti-oxidative and intestinal barrier strengthening properties of this strain.

The main beneficial properties of Lactobacillus plantarum DSM 33464 are summarized in Table 1.

TABLE 1
summary of beneficial properties of
L. plantarum strain DSM 33464

| Beneficial properties | In vitro | In vivo |
|---|---|---|
| Lead binding to the cell surface and | X | |
| Survival to gastric and instestinal fluid | X | |
| Adhesion to intestinal cells | X | |
| Exopolysaccharide production | X | |
| Inhibition of pro-inflammatory cytokines | | X |
| interleukin (IL)-1β, IL-6, IL-17F, and tumor | | |
| necrosis factor (TNF)-α | | |
| Antioxidative properties | | X |
| Intestinal barrier strenghtening | | X |
| Reduction of blood lead level in mice | | X |
| Reduction of lead levels in kidney, liver, and | | X |
| brain tissues in mice | | |
| Induction of hepatic bile acid synthesis and | | X |
| stimulation of bile acid secretion (an additional | | |
| mechanism that can lead to heavy metal | | |
| excretion from the GI tract) | | |

In a preferred embodiment, the Lactobacillus cells bind to the heavy metal cation such as the lead ion (Pb²¹) or cadmium ion (Cd²⁺).

In another or a further preferred embodiment, the Lactobacillus cells bind to heavy metal cations in vitro. The in vitro binding of the heavy metal cations to dried Lactobacillus cells is detected using a lead binding assay known to the person skilled in the art. In an embodiment, the lead binding assay includes incubating the dried Lactobacillus cells with a medium containing heavy metal cations. The incubated Lactobacillus cells are centrifuged to separate the Lactobacillus cells and heavy metal cations. After centrifugation, the supernatant is collected as the supernatant contains the heavy metal cations. The heavy metal cations concentration is measured in the supernatant. The heavy metal cations concentration can be measured using colorimetry e.g. using a Supelco Kit as described in Example 1, or any other measuring technique know in the art. For example, the remaining heavy lead cations concentration in the supernatant can be measured using Inductively Coupled Plasma (ICP) spectroscopy.

In another preferred embodiment, the Lactobacillus cells bind to heavy metal cations in vivo. The in vivo binding of heavy metal cations to dried Lactobacillus cells is detected by measuring the reduction of heavy metals in blood as well as in different organs (kidney, brain, liver, bones) Non-limiting examples of a Lactobacillus include: Lactobacillus delbrueckii, Lactobacillus acetotolerans, Lactobacillus achengensis, Lactobacillus acidifarinae, Lactobacillus acidipiscis, Lactobacillus acidophilus, Lactobacillus agilis, Lactobacillus algidus, Lactobacillus alimentarius, Lactobacillus allii, Lactobacillus alvi, Lactobacillus amylolyticus, Lactobacillus amylophilus, Lactobacillus amylotrophicus, Lactobacillus amylovorus, Lactobacillus angrenensis, Lactobacillus animalis, Lactobacillus antri, Lactobacillus apinorum, Lactobacillus apis, Lactobacillus apodemi, Lactobacillus aquaticus, Lactobacillus argentoratensis, Lactobacillus arizonensis, Lactobacillus aviarius, Lactobacillus backii, Lactobacillus baiquanensis, Lactobacillus bambusae, Lactobacillus baoqingensis, Lactobacillus bavaricus, Lactobacillus bayanensis, Lactobacillus bifermentans, Lactobacillus binensis, Lactobacillus bobalius, Lactobacillus bombi, Lactobacillus bombicola, Lactobacillus bombintestini, Lactobacillus brantae, Lactobacillus brevis, Lactobacillus buchneri, Lactobacillus bulgaricus, Lactobacillus cacaonum, Lactobacillus camelliae, Lactobacillus capillatus, Lactobacillus carnis, Lactobacillus casei, Lactobacillus catenaformis, Lactobacillus caucasicus, Lactobacillus caviae, Lactobacillus cellobiosus, Lactobacillus cerevisiae, Lactobacillus ceti, Lactobacillus chiayiensis, Lactobacillus coleohominis, Lactobacillus colini, Lactobacillus collinoides, Lactobacillus composti, Lactobacillus concavus, Lactobacillus confusus, Lactobacillus coryniformis, Lactobacillus crispatus, Lactobacillus crustorum, Lactobacillus curieae, Lactobacillus curtus, Lactobacillus curvatus, Lactobacillus cypricasei, Lactobacillus daoliensis, Lactobacillus daowaiensis, Lactobacillus daqingensis, Lactobacillus dextrinicus, Lactobacillus diolivorans, Lactobacillus divergens, Lactobacillus dongliensis, Lactobacillus durianis, Lactobacillus enshiensis, Lactobacillus equi, Lactobacillus equicursoris, Lactobacillus equigenerosi, Lactobacillus fabifermentans, Lactobacillus faecis, Lactobacillus farciminis, Lactobacillus farraginis, Lactobacillus ferintoshensis, Lactobacillus fermentum, Lactobacillus floricola, Lactobacillus florum, Lactobacillus formosensis, Lactobacillus fornicalis, Lactobacillus fructivorans, Lactobacillus fructosus, Lactobacillus frumenti, Lactobacillus fuchuensis, Lactobacillus fujinensis, Lactobacillus furfuricola, Lactobacillus futsaii, Lactobacillus fuyuanensis, Lactobacillus gallinarum, Lactobacillus gannanensis, Lactobacillus garii, Lactobacillus gasseri, Lactobacillus gastricus, Lactobacillus ghanensis, Lactobacillus gigeriorum, Lactobacillus ginsenosidimutans, Lactobacillus gorillae, Lactobacillus graminis, Lactobacillus halodurans, Lactobacillus halotolerans, Lactobacillus hammesii, Lactobacillus hamsteri, Lactobacillus harbinensis, Lactobacillus hayakitensis, Lactobacillus hegangensis, Lactobacillus heilongjiangensis, Lactobacillus helsingborgensis, Lactobacillus helveticus, Lactobacillus herbarum, Lactobacillus heterohiochii, Lactobacillus hilgardii, Lactobacillus hokkaidonensis, Lactobacillus hominis, Lactobacillus homohiochii, Lactobacillus hordei, Lactobacillus huachuanensis, Lactobacillus huananensis, Lactobacillus hulanensis, Lactobacillus hulinensis, Lactobacillus iners, Lactobacillus ingluviei, Lactobacillus insicii, Lactobacillus intestinalis, Lactobacillus iwatensis, Lactobacillus ixorae, Lactobacillus jensenii, Lactobacillus jiayinensis, Lactobacillus jidongensis, Lactobacillus jinshani, Lactobacillus jixianensis, Lactobacillus johnsonii, Lactobacillus kaifaensis, Lactobacillus kalixensis, Lactobacillus kandleri, Lactobacillus kedongensis, Lactobacillus kefir, Lactobacillus kefiranofaciens, Lactobacillus kefirgranum, Lactobacillus keshanensis, Lactobacillus kimbladii, Lactobacillus kimchicus, Lactobacillus kimchiensis, Lactobacillus kimchii, Lactobacillus kisonensis, Lactobacillus kitasatonis, Lactobacillus koreensis, Lactobacillus kosoi, Lactobacillus kullabergensis, Lactobacillus kunkeei, Lactobacillus lactis, Lactobacillus leichmannii, Lactobacillus lindianensis, Lactobacillus lindneri, Lactobacillus malefermentans, Lactobacillus mali, Lactobacillus maltaromicus, Lactobacillus manihotivorans, Lactobacillus mellifer, Lactobacillus mellis, Lactobacillus melliventris, Lactobacillus metriopterae, Lactobacillus micheneri, Lactobacillus mindensis, Lactobacillus minor, Lactobacillus minutus, Lactobacillus mishanensis, Lactobacillus mixtipabuli, Lactobacillus modestisalitolerans, Lactobacillus mucosae, Lactobacillus mudanjiangensis, Lactobacillus mulanensis, Lactobacillus mulengensis, Lactobacillus mulieris, Lactobacillus murinus, Lactobacillus musae, Lactobacillus nagelii, Lactobacillus namurensis, Lactobacillus nangangensis, Lactobacillus nantensis, Lactobacillus nasuensis, Lactobacillus nenjiangensis, Lactobacillus nodensis, Lactobacillus nuruki, Lactobacillus odoratitofui, Lactobacillus oeni, Lactobacillus oligo, Lactobacillus oris, Lactobacillus oryzae, Lactobacillus otakiensis, Lactobacillus ozensis, Lactobacillus panis, Lactobacillus panisapium, Lactobacillus pantheris, Lactobacillus parabrevis, Lactobacillus parabuchneri, Lactobacillus paracasei, Lactobacillus paracollinoides, Lactobacillus parafarraginis, Lactobacillus paragasseri, Lactobacillus parakefiri, Lactobacillus paralimentarius, Lactobacillus paraplantarum, Lactobacillus pasteurii, Lactobacillus paucivorans, Lactobacillus pentosiphilus, Lactobacillus pentosus, Lactobacillus perolens, Lactobacillus pingfangensis, Lactobacillus piscicola, Lactobacillus plajomi, Lactobacillus plantarum, Lactobacillus pobuzihii, Lactobacillus pontis, Lactobacillus porci, Lactobacillus porcinae, Lactobacillus psittaci, Lactobacillus quenuiae, Lactobacillus raoultii, Lactobacillus rapi, Lactobacillus rennini, Lactobacillus reuteri, Lactobacillus rhamnosus, Lactobacillus rimae, Lactobacillus rodentium, Lactobacillus rogosae, Lactobacillus rossiae, Lactobacillus ruminis, Lactobacillus saerimneri, Lactobacillus sakei, Lactobacillus salitolerans, Lactobacillus salivarius, Lactobacillus salsicarnum, Lactobacillus sanfranciscensis, Lactobacillus saniviri, Lactobacillus satsumensis, Lactobacillus secaliphilus, Lactobacillus selangorensis, Lactobacillus senioris, Lactobacillus senmaizukei, Lactobacillus sharpeae, Lactobacillus shenzhenensis, Lactobacillus sicerae, Lactobacillus silagei, Lactobacillus silagincola, Lactobacillus siliginis, Lactobacillus similis, Lactobacillus sobrius, Lactobacillus songbeiensis, Lactobacillus songhuajiangensis, Lactobacillus spicheri, Lactobacillus suantsaicola, Lactobacillus suantsaii, Lactobacillus suantsaiihabitans, Lactobacillus sucicola, Lactobacillus suebicus, Lactobacillus suibinensis, Lactobacillus sunkii, Lactobacillus suntoryeus, Lactobacillus taiwanensis, Lactobacillus tangyuanensis, Lactobacillus terrae, Lactobacillus thailandensis, Lactobacillus thermotolerans, Lactobacillus timberlakei, Lactobacillus timonensis, Lactobacillus tongiiangensis, Lactobacillus trichodes, Lactobacillus tucceti, Lactobacillus uli, Lactobacillus ultunensis, Lactobacillus uvarum, Lactobacillus vaccinostercus, Lactobacillus vaginalis, Lactobacillus versmoldensis, Lactobacillus vespulae, Lactobacillus vini, Lactobacillus viridescens, Lactobacillus vitulinus, Lactobacillus wasatchensis, Lactobacillus wuchangensis, Lactobacillus xiangfangensis, Lactobacillus xujianguonis, Lactobacillus xylosus, Lactobacillus yamanashiensis, Lactobacillus yichunensis, Lactobacillus yilanensis, Lactobacillus yonginensis, Lactobacillus zeae, Lactobacillus zhachilii, Lactobacillus zhaodongensis, Lactobacillus zhaoyuanensis, Lactobacillus zhongbaensis, Lactobacillus zymae, Lactobacillus sp.

The non-limiting examples of a Lactobacillus also include any proposed reclassification of the genus Lactobacillus such as Lactobacillus delbrueckii genus, Paralactobacillus, Holzapfelia, Amylolactobacillus, Bombilactobacillus, Companilactobacillus, Lapidilactobacillus, Agrilactobacillus, Schleiferilactobacillus, Loigolactobacilus, Lacticaseibacillus, Latilactobacillus, Dellaglioa, Liquorilactobacillus, Ligilactobacillus, Lactiplantibacillus, Furfurilactobacillus, Paucilactobacillus, Limosilactobacillus, Fructilactobacillus, Acetilactobacillus, Apilactobacillus, Levilactobacillus, Secundilactobacillus and Lentilactobacillus genera as proposed in “A taxonomic note on the genus Lactobacillus: Description of 23 novel genera, emended description of the genus Lactobacillus Beijerinck 1901, and union of Lactobacillaceae and Leuconostocaceae”—published in International Journal of Systematic and Evolutionary Microbiology, Volume 70, Issue 4”.

### Use

In a preferred embodiment of the process, the dried Lactobacillus cells is a lead binding product. The lead binding product removes lead from the gastro-intestinal (GI) tract of the human body. The lead binding product can be used as probiotics or therapeutics to treat or manage negative health outcomes due to lead exposure in humans. The lead binding product is in a further or alternative embodiment used to reduce the level of heavy metals in the body such as in the human body. In a yet further or alternative embodiment, the lead binding product is used to eliminate heavy metals in the body such as the human body. In a still further or alternative embodiment, the lead binding product facilitates decreased absorption of lead. In a yet still further or alternative embodiment, the lead binding product is used as a dietetic food or a food supplement. In an embodiment, the lead binding product used as a dietetic food or a food supplement is for a special medical purpose. In a further embodiment, the specific medical purpose is the dietary management of lead uptake in the body.

The invention further relates to a pharmaceutical, food, functional food, dietetic food, dietary food, dietary supplement, medical device and/or therapeutic composition comprising a physiologically effective dose of the dried Lactobacillus cells according to the invention and a physiologically compatible carrier. The pharmaceutical compositions are compositions which serve therapeutic and/or prophylactic purposes, which in addition to dried Lactobacillus cells according to the invention, e.g. comprise adjuvants and/or excipients that are common in pharmaceutical compositions. The dietary compositions within the meaning of the present invention are compositions which, in addition to the dried Lactobacillus cells according to the invention, comprise a food, foodstuff and/or dietary supplement.

The invention further relates to the use or application of the dried Lactobacillus cells according to the invention for producing a pharmaceutical or dietary composition, or a pharmaceutical product or a dietary supplement, comprising the dried Lactobacillus cells or a pharmaceutical or dietary composition, in particular for the management of negative health outcomes associated with lead exposure.

Particular embodiments of the present disclosure are described in the following numbered paragraphs:
1. A process for producing dried Lactobacillus cells comprising the steps:
   a. fermenting Lactobacillus cells in a fermentation medium;
   b. obtaining a fermentation product comprising the Lactobacillus cells;
   c. optionally concentrating the fermentation product;
   d. adjusting the fermentation product to a pH range between pH 8 and 11;
   e. drying the pH adjusted fermentation product;
      wherein step d. is optionally applied before step c.
2. The process of paragraph 1, wherein the drying is freeze drying or spray drying.
3. The process of any one of the preceding paragraphs, wherein the freeze drying is carried out at a temperature ranging between −60° C. and +50° C. and for a time ranging between 12 hours to 100 hours, preferably at temperature between −45° C. and +30° C. and for a time ranging between 24 hours to 96 hours, or between −30° C. and +20° C. for about 66 hours.
4. The process of any one of the preceding paragraphs, wherein the Lactobacillus cells are dead.
5. The process of any one of the preceding paragraphs, wherein the Lactobacillus cells are heat-killed
6. The process of any one of the preceding paragraphs, wherein the Lactobacillus cells are Lactobacillus plantarum cells.
7. The process of any one of the preceding paragraphs, wherein Lactobacillus plantarum are deposited as DSM 33464.
8. The process of any one of the preceding paragraphs, wherein the Lactobacillus cells bind to heavy metal cations in vitro and/or in vivo.
9. The process of any one of the preceding paragraphs, wherein the heavy metal cation is Lead (Pb²⁺) or Cadmium (Cd²⁺)
10. The process of any one of the preceding paragraphs, wherein the dried Lactobacillus cells is a lead binding product.
11. The process of any one of the preceding paragraphs, wherein the dried Lactobacillus cells removes lead from the gastrointestinal tract of a human body.
12. The process of any one of the preceding paragraphs, wherein the fermentation product is adjusted to about pH 8 to 10, about pH 8.5 to 10, about pH 9 to 10, about pH 9, about pH 9.5, or about pH 10.
13. The process of any one of the preceding paragraphs, wherein the fermentation product is adjusted to about pH 9, about pH 9.5 or about pH 10.
14. The process of any one of the preceding paragraphs, wherein the in vitro binding of the heavy metal cations to dried Lactobacillus cells is detected by:
   a) incubating the dried Lactobacillus cells with a medium containing heavy metal cations,
   b) centrifuging the incubated Lactobacillus cells to separate the Lactobacillus cells and heavy metal cations,
   c) collecting the supernatant, and
   d) measuring the heavy metal cations concentration in the supernatant.
15. The process of any one of the preceding paragraphs, wherein the in vivo binding of the heavy metal cations to dried Lactobacillus cells is detected by measuring the reduction of the heavy metal cations in the blood and/or organs.
16. The process of any one of the preceding paragraphs, wherein the binding of heavy metal cations to the dried Lactobacillus cells is higher compared to binding of heavy metal cations to dried Lactobacillus cells prepared at pH less than 8 or more than 11.
17. The process of any one of the preceding paragraphs, wherein the binding of essential minerals by the dried Lactobacillus cells is such that the binding is not leading to deficiency of essential minerals in the body.
18. The process of any one of the preceding paragraphs, wherein the Lactobacillus cells are concentrated by centrifugation at step c. of claim 1.
19. The process of any one of the preceding paragraphs, wherein the fermentation product or concentrated fer-mentation product contains one or more additives.
20. The process of any one of the preceding paragraphs, wherein the one or more additives is a cryoprotectant and/or a stabilizer.
21. The process of any one of the preceding paragraphs, wherein the cryoprotectant is glucose, lactose, raffinose, sucrose, trehalose, adonitol, glycerol, mannitol, methanol, polyethylene glycol, propylene glycol, ribitol, alginate, bovine serum albumin, carnitine, citrate, cysteine, dextran, dimethyl sulphoxide, sodium glutamate, glycine betaine, glycogen, hypotaurine, peptone, polyvinyl pyrrolidone, or taurine, mammalian milk oligosaccharides, chitin, chitosan, casein, yeast, yeast extract, single cell protein, mycoproteins, other disaccharides or polysaccharides, or mixtures thereof.
22. The process of any one of the preceding paragraphs, wherein the cryoprotectant is a dextrin.
23. Lactobacillus cells obtained from the process according to any one of the preceding paragraphs.
24. Lactobacillus cells dried in the process according to any one of paragraphs 1 to 22.

### Examples

The following examples are not intended to be a detailed catalogue of all the different ways in which the present disclosure may be implemented or of all the features that may be added to the present disclosure. Subjects skilled in the art will appreciate that numerous variations and additions to the various embodiments may be made without departing from the present disclosure. Hence, the following descriptions are intended to illustrate some particular embodiments of the invention and not to exhaustively specify all permutations, combinations and variations thereof.

Unless otherwise indicated, the percentages set forth in the following examples are by weight, based upon the total weight of the composition.

### Deposit of Biological Material

The following biological material has been deposited under the terms of the Budapest Treaty with Leibniz-Institut DSMZ—Deutsche Sammlung von Mikroorganismen und Zellkulturen GmbH (DSM), Inhoffenstr. 7 B, D-38124 Braunschweig, Germany, and given the following accession number:

| Deposit | Accession Number | Date of Deposit | Origin |
|---|---|---|---|
| SmartGuard | DSM 33464 | 5 Mar. 2020 | China |

The strain has been deposited under conditions that assure that access to the culture will be available during the pendency of this patent application to one determined by foreign patent laws to be entitled thereto. The deposit represents a substantially pure culture of the deposited strain. The deposit is available as required by foreign patent laws in countries wherein counterparts of the subject application, or its progeny are filed. However, it should be understood that the availability of a deposit does not constitute a license to practice the subject invention in derogation of patent rights granted by governmental action

### Example 1—Investigation of Reduction of Blood Lead Level in Acute Mouse Model

### Study Design

The study investigated the ability of orally administered L. plantarum DSM 33464 to reduce the gastrointestinal uptake of orally ingested PbAc2 and thereby lowering the lead blood levels in mice during an acute lead toxicity challenge. In this study, C57BL/6 male mice (4-6 weeks of age) were challenged with a single oral dose of PbAc2 (100 mg/kg body weight/day) and used in 2 different studies with 5-10 animal/studies. The levels of lead challenge used could be translated to the level of lead potentially ingested in humans that are exposed to lead through contaminated food and water. Furthermore, the study aimed to demonstrate effect on intestinal barrier by analyzing expression of four tight junction proteins in samples from the small intestine.

At day −1, the mice were either treated prophylactically with L. plantarum DSM 33464 (1×10⁹ CFU/mouse) or with the chelating agent, dimercaptosuccinic acid (DMSA) (50 mg/kg, dissolved in protectant solution). The disease and healthy control groups received a PBS dosage at the same time. Then on day 0, 1 and 2 all mice were treated with either PBS, L. plantarum DSM 33464 or DMSA one hour prior to a lead acetate treatment of 100 mg/kg. The healthy control received saline instead of lead.

Feces samples from the mice were collected after the first lead gavage on day 0 and recorded as 0 h feces sample, and then at 12 h, 24 h, 36 h, 48 h, 52 h, 56 h, 60 h, 66 h, 72 h. Mice were anesthetized with ether, and blood were collected by heart puncture. After euthanasia, liver, kidney, bone, small intestine and brain tissues were collected from all mice.

0.2 ml blood or 0.2 g of liver, brain, kidney, and feces from each mouse were collected separately, and then added into a dissolution tank with 5 ml of nitric acid for cold digestion overnight. A microwave digestion system was then used for complete digestion. The resulting mixture was then diluted to 10 ml with deionized water, and the lead content was measured using an Inductively Coupled Plasma Mass Spectrometry (ICP-MS).

The intestinal barrier plays a crucial role in limiting Pb absorption and exposure to Pb damage the tight junctions in the intestines leading to disruption of the intestinal barrier and further amplification of Pb absorption and toxicity. qPCR analysis of tight junction proteins in the small intestine of samples.

### Results

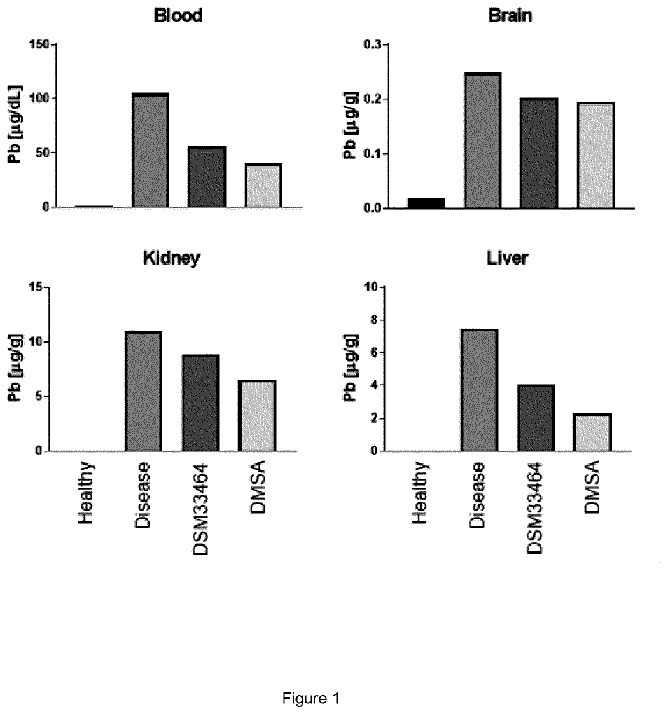

The results of the study showed (FIG. 1) that the L. plantarum DSM 33464 significantly reduced the lead content in blood, bone, brain, liver, kidney in mice dosed with 100 ppm Pb and this effect was comparable to the effect seen in mice treated with the positive drug control (DMSA). A possible trend for a higher excretion on lead excreted in fecal content with L. plantarum DSM 33464 or DMSA compare to the control group.

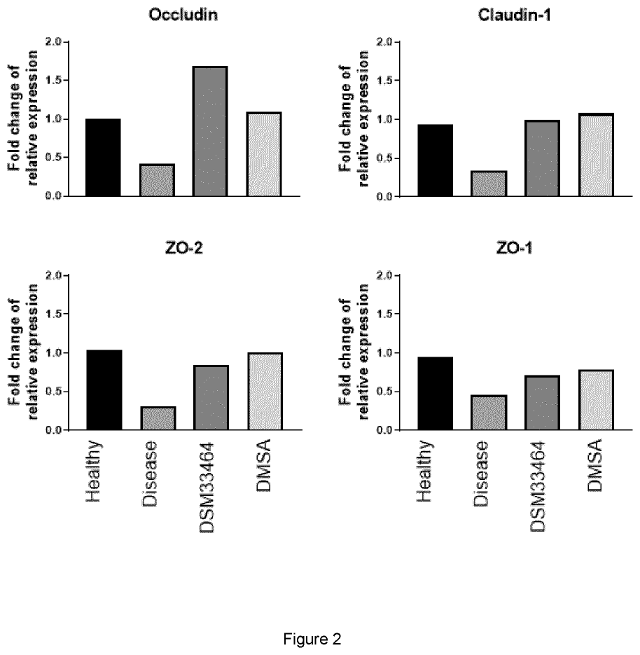

By performing qPCR analysis of tight junction proteins in the small intestine of samples, it was shown in FIG. 2 that L. plantarum DSM 33464 normalize expression levels of the tight junction proteins occluding, claudin-1 and ZO-2. Expression levels of ZO-1 was not improved by SmartGuard or DMSA.

In conclusion the study confirmed that L. plantarum DSM 33464 reduces lead absorption in the intestine and thereby lowers the lead content in blood, brain, liver and kidney as well as improve the barrier integrity of the small intestine.

### Example 2—Improved Pb²⁺ Adsorption of Freeze-Dried Lactobacillus Cells Derived from Different Fermentation (Down-Stream) Processes

### Samples:

Three samples were prepared:
HH10F39D02: No pH adjustment before freeze drying (neutral pH)
HH10F39D04: pH adjusted to pH9 before freeze drying process
HH10F39D05: pH adjustment to pH10 before freeze drying process

### Fermentation:

### Storage of Strains, Cryostock

The Lactobacillus strains were stored in the frozen state as cryostocks. 1 ml of a culture cultured up to the stationary phase (OD₆₀₀/ml 4-8) in MRS medium (55 g/l, pH 6.5; Difco, USA) was mixed with 500 μl of a 50% (v/v) sterile glycerin solution, and the mixture was frozen at −80° C.

### Preculture Media

25 g/L yeast extract NuCel 582 (Procelys), 2 g/L di-ammonium hydrogen citrate, 5 g/L sodium acetate, 0.1 g/L magnesium sulphate heptahydrate, 0.05 g/L manganese(II)sulfate monohydrate, 2 g/L dipotassium hydrogen phosphate, 1/L tween 80, 20 g/L glucose.

### Main Culture Media

30 g/l yeast extract Nucel 582, 0,022 g/L manganese(II) sulfate monohydrate, 1 g/L tween80, 40 g/L glucose, 40 g/L fructose

Preculture 1 was prepared from the preculture media which was inoculated with 2% (v/v) of cryostock of the strain Lactobacillus plantarum DSM33464 and cultivated at 37° C. for 15-16 hours. Subsequent preculture 2 was prepared by inoculating the preculture media with 2% of preculture 1 and cultivated for 7.5-8 hours at 37° C. Fermenters were autoclaved with the main culture media. Glucose and fructose solutions (60%) were added separately to the main culture media after autoclaving. A fermenter was cooled down to 5° C. and inoculated with 3% (v/v) of preculture 2. For the main fermentation, the fermenter was heated up to 37° C. and run for 12-16.5 hours. Prior to harvesting, the fermenter was cooled down to 5° C. for 30 minutes.

### Determination of CFU/Ml in Fermentation Broth

A decadic dilution series with 1×PBS/NaCl-Peptone was prepared until 10⁻⁶. A volume of 50 μL was plated on MRS agar plates with spiral plater in duplicates per dilution (log mode 50 μL, 2, 1/1). After incubation (24-48 hours, 37° C., anaerobic conditions), the colony forming units (CFU) were determined via the Colony counter.

### Harvest of Fermentation Broth

A volume of 300 mL per sample was centrifuged (4.000×g, 15 min, 4° C.) and the supernatant was discarded. After determination of the cell wet weight (CWW), a pellet was resuspended in 20% (w/w) Nutriose FM06 (Roquette) solution that was added in a 2:1 ratio on a dry matter base. The pH of each sample was adjusted to the respective value with 25% NH₃. Samples that were not yet adjusted were stored at 5° C.

| Sample | Intended pH adjustment | Actual pH adjusted |
|---|---|---|
| HH10F39D02 | — | — |
| HH10F39D04 | 9.0 | 8.96 |
| HH10F39D05 | 10.0 | 9.95 |

The adjusted samples were transferred into a product dish and frozen at −80° C. for 24 hours. Electrodes for measuring the temperature and degree of dryness were added to one sample.

Downstream processing (DSP) and Freeze Drying

The following program was used for lyophilization of the frozen samples:
1. Warm up freeze dryer (shelf temp. −40° C.)
2. Main drying 0.22 mbar, −20° C., for 24 hours
3. Main drying 0.22 mbar, 1 hour ramp to 0° C.
4. Main drying 0.22 mbar, 0° C. for 34 hours
5. Final drying 0.02 mbar, 1 hour ramp to 20° C.
6. Final drying 0.02 mbar, 20° C. for 6 hours

After 66 hours, the powder was homogenized and stored in vacuum packed alu-bags for flow cytometric analysis and Pb² binding Assay. Additionally, the water activity (aW) was measured of each sample.

### 1. Determination of CFU in Powder

In order to determine the CFU of the freeze-dried powder, 2×100 mg of freeze-dried powder were dissolved in 9.9 mL of PBS 1×/NaCl-Peptone and were incubated for 15 minutes at room temperature. The dissolved powders were further diluted until 10⁻⁵ (equals 10⁻⁷ in total) and plated on MRS-Agar via a spiral plater. After incubation for 24-48 hours at 37° C. under anaerobic conditions, the CFU/g was determined by colony counter.

### Lead Binding-Assay

### Reagents

Lead (II) acetate trihydrate, Pb(CH₃CO₂)₂·3H₂O, Sigma-Aldrich #316512
Supelco Kit 1.09717.0001
Sodium acetate trihydrate, Sigma-Aldrich #S8625
Ultra-pure water

### Sample Preparation

100 mg of freeze-dried powder was resuspended in 20 mL of ultra-pure water in 50 mL Falcon tubes
The tubes were vortexed for minimum 10 seconds
After vortexing, the tubes were centrifuged at 4000×g for 10 minutes
Supernatant was removed without disturbing the pellet
The pellet was resuspended in 20 mL of 50 mM acetate buffer at pH 5.6 by vortexing for minimum 10 seconds to provide a cell preparation

### Incubation Assay

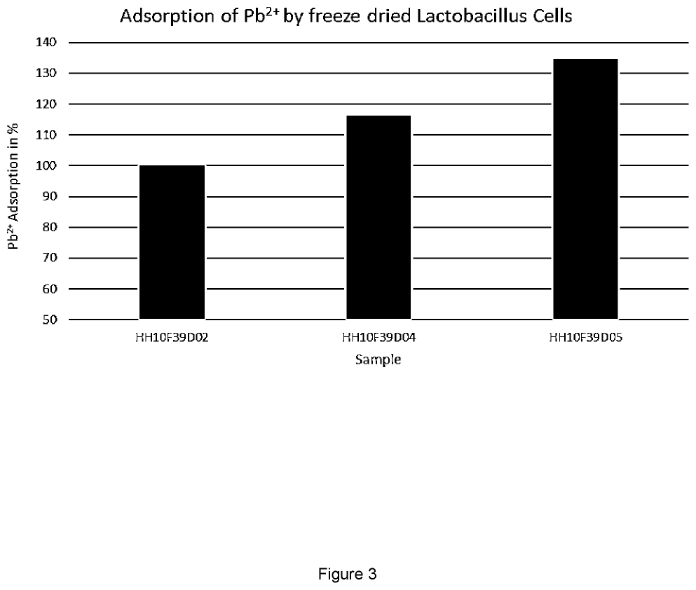

Cell/Pb²⁺-acetate mixture [150 μL of cell suspension, 750 μL of ultra-pure water and 100 μL of Pb²⁺-acetate solution (1450 M)] were transferred to 24-well plate
Plate was covered with an adhesive seal foil
The cell/Pb²-acetate mixture was incubated for 1 hour at 37° C. while shaking at 150 rpm in incubator
The incubated mixture was centrifuged at 4500×g for 10 minutes
The supernatant was collected by a pipette
50 μL of collected supernatant was transferred to 96 well round bottom plate and diluted 1:6 with ultra pure water
   Colorimetric Assay with Supelco Kit 1.09717.0001
10 μL of reagent Pb-1 was transferred into flat bottom plate
Thereafter, 10 μL of reagent Pb-2 was added to the flat bottom plant and mixed with a pipette
160 μL of sample was added and mixed with a pipette
A blank with 160 μL pure water was included
A dilution series of Pb-Acetate 0-1450 μM was included for calculation of the calibration curve
OD was measured at 525 nm
   The results are illustrated in FIG. 3 and show Pb²⁺ adsorption of freeze-dried Lactobacillus cells derived from different fermentation-(down-stream) processes. The results demonstrate that the Lactobacillus cells adsorb Pb²⁺ where the Lactobacillus cells which were pH adjusted to pH9 or pH10 before freeze drying process have a higher level of Pb² adsorption compared to Lactobacillus cells which were freeze dried without any pH adjustment (having neutral pH).

### 2. Enumeration of Lactobacillus Cells by Flow Cytometry

### Initializing Flow Cytometer

1.1 0.5% Sodium Hypochloride solution (10% Bleach) was prepared
1 mL 12-15% Sodium hypochloride solution (stored in 4° C. fridge; black-walled 50 ml greiner tube) was added to 19 ml dH2O in a 50 ml greiner tube
On each day of analysis, this solution was prepared fresh

### 1.2 Startup

Turn on the PC
Switch on 1. Auto Sampler, 2. Attune NxT
Start Attune NxT Software
Press shortcut: “Performance Test”
Press: Startup (Startup takes around 2 min)

### 1.3 Performance Test

3 drops of Performance tracking beads were dropped into a flow cytometer tube, and 2 ml Focusing fluid and vortex was added for 1 see
The tube was placed into the sample tube lift and lifted up
“Run Performance test” was pressed (approximately 4 minutes)
As the Performance test was passed, only green ticks appeared on the Performance test report;
“Main Menu” was selected

### 2.1 Experiment Setup

In “Main Menu”, shortcut “New Experiment from Template” was pressed
“Enumeration Microbes SYTO13+PI” was selected, thereafter, “Next” and “Finish” was selected
On created plate experiment, “Experiment Explorer” was selected
Experiment and Plate was renamed; Date and ELN-No. was used to name the experiment
The tab “Heat Map” was selected to define plate layout; well positions were defined, followed by selecting “New Sample” and the defined wells were added to a group (each group represents a sample dilution (e.g. 1E-2)
The tab “Sample List” was used to name samples

### 3.1 Start Flow Cytometric Analysis

focusing fluid and waste container was checked, if refilled or emptied
The plate was loaded into Auto Sampler after completed staining procedure without the lid
“Record plate” was selected in window “Collection Panel”

### 4.1 Shut Down Attune NxT

After completion of analysis, the device was shutdown by loading an empty, clean 96 well round bottom MTP into Auto Sampler
Sanitize sip was performed with 1:3 diluted cell flow cleaning solution (diluted in ultra pure water)
3 ml of 0.5% Sodium Hypochloride solution was added into a flow cytometer tube
The tube was placed into the sample tube lift and lifted up
“Shutdown” was selected within the tab “Instrument” and thereafter “Thorough” was selected

### Sample Preparation and Staining Procedure

### 1.1 Sample Preparation (Fermentation Samples)

Sterile filtered PBS (w/o Ca and Mg) was used to dilute sample decadal in 96 deepwell plate (900 μl PBS+100 μl previous dilution) to 1E-5
200 μl/well of cell suspension was transferred according to layout to a 96 well round bottom plate
Dilutions 1E-2-1E-5 were analyzed
This dilution range was appropriate for fermentation samples up to OD600=30

### 1.2 Sample Preparation (Stability Samples)

Sterile filtered PBS (w/o Ca and Mg) was used to dilute sample decadal in 96 deepwell plate (900 μl PBS+100 μl previous dilution) to 1E-7 or prepared dilutions for CFU counts (Peptone-NaCl solution) were used
200 μl/well of cell suspension was transferred according to layout 96 well round bottom plate
Dilutions 1E-4-1E-7 were analyzed for estimated total cell count of up to 5E+11 cells/mL

### 2. Staining Procedure

A premix of SYTO13 and PI working solution was prepared according to following table in 1.5 mL Eppendorf tubes and by vortexing

| | | | | Premix dye |
|---|---|---|---|---|
| | | | | application volume |
| | | Preparation | Premix preparation of | for 200 μl/well cell |
| Dye | Stock solution | Working solution | Syto13 and PI | suspension |
| PI | 1 mg/mL in H2O | 24 μl stock solution + | Mix 590 μl of each | 10 μl |
| (powder) | (20 mM) (can be stored | 576 μl PBS | working solution in 1.5 | |
| | for 2 weeks at 4° C.) | | ml tube; | |
| Syto13 | 5 mM in DMSO | 5 μl stock solution + | Aliquot 8x 140 μl in a | |
| | (stored at −20° C.) | 620 μl PBS | stripe with 8 PCR tubes | |

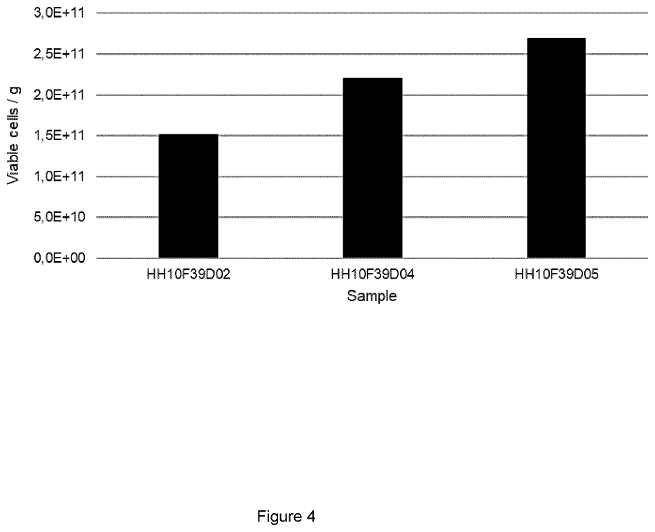

10 μl/well PI working solution (not resuspend, content was only ejected into well; new pipette was used for each well) was added
Each well was mixed 3 times with green multichannel pipette (100 l)
The tubes were incubated for 15 minutes at room temperature in the dark with a lid
Within 45 minutes the tubes were analyzed
   Cell viability of freeze-dried Lactobacillus cells derived from different fermentation-(down-stream) processes was determined using flow cytometry and is illustrated in FIG. 4. The experiment demonstrates that the Lactobacillus cells which were pH adjusted to pH9 or pH10 before freeze drying process have improved cell viability compared to Lactobacillus cells which were freeze dried without any pH adjustment (having neutral pH).

### Example 3—High Resolution Microscopy of Pb²⁺ Adsorption by Lactobacillus Cells

### Samples:

HH10F39D02: Freeze dried cells with no pH adjustment before freeze drying (neutral pH)
HH10F39D04: Freeze dried cells with pH adjustment to pH 9 before freeze drying

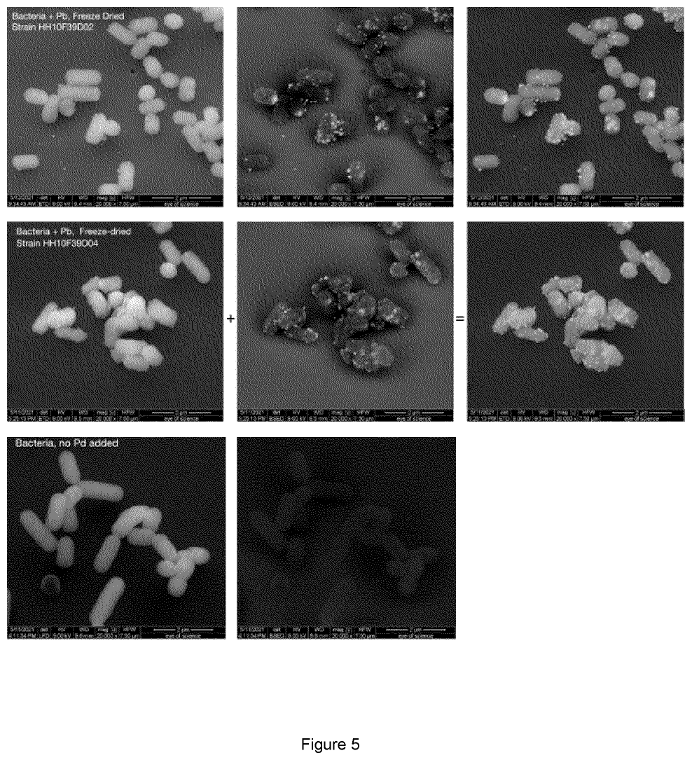

Pb²⁺ adsorption by the Lactobacillus cells was shown using high resolution microscopy (FIG. 5). When samples (HH10F39D02 and HH10F39D04) as defined in Example 1 were analyzed using high resolution microscopy, the Lactobacillus cells which were pH adjusted to 9 before freeze drying process had improved level of Pb² adsorption compared to Lactobacillus cells which were freeze dried without any pH adjustment (having neutral pH).

1: A process for producing dried Lactobacillus cells comprising the steps:
a. fermenting Lactobacillus cells in a fermentation medium;
b. obtaining the fermentation product comprising the Lactobacillus cells;
c. optionally concentrating the fermentation product;
d. adjusting the fermentation product to a pH range between pH 8 and 11;
e. drying the pH adjusted fermentation product;
wherein step d. is optionally applied before step c.

2: The process according to claim 1, wherein the drying is freeze drying or spray drying.

3: The process according to claim 1, wherein the freeze drying is carried out at a temperature ranging between −60° C. and −100° C. and for a time ranging between 20 hours to 30 hours.

4: The process according to claim 1, wherein the Lactobacillus cells are Lactobacillus plantarum cells.

5: The process according to claim 4, wherein Lactobacillus plantarum is deposited as DSM 33464.

6: The process according to claim 1, wherein the Lactobacillus cells bind to heavy metal cations in vitro and/or in vivo.

7: The process according to claim 1, wherein the heavy metal cation is Lead (Pb2+) or Cadmium (Cd2+).

8: The process according to claim 1, wherein the fermentation product is adjusted to about pH 8 to 10.

9: The process according to claim 1, wherein the in vitro binding of the heavy metal cations to dried Lactobacillus cells is detected by:
a) incubating the dried Lactobacillus cells with a medium containing heavy metal cations,
b) centrifuging the incubated Lactobacillus cells to separate the Lactobacillus cells and heavy metal cations,
c) collecting the supernatant, and
d) measuring the heavy metal cations concentration in the supernatant.

10: The process according to claim 1, wherein the in vivo binding of the heavy metal cations to dried Lactobacillus cells is detected by measuring the reduction of the heavy metal cations in the blood and/or organs.

11: The process according to claim 1, wherein the binding of heavy metal cations to the dried Lactobacillus cells is higher compared to binding of heavy metal cations to dried Lactobacillus cells prepared at pH less than 8 or more than 11.

12: The process according to claim 1, wherein the Lactobacillus cells are concentrated by centrifugation at step c. of claim 1.

13: The process according to claim 1, wherein the fermentation product or concentrated fermentation product contains one or more additives such as a cryoprotectant and/or a stabilizer.

14: The process according to claim 1, wherein the cryoprotectant is glucose, lactose, raffmose, sucrose, trehalose, adonitol, glycerol, mannitol, methanol, polyethylene glycol, propylene glycol, ribitol, alginate, bovine serum albumin, carnitine, citrate, cysteine, dextrin, dimethyl sulphoxide, sodium glutamate, glycine betaine, glycogen, hypotaurine, peptone, polyvinyl pyrrolidone, taurine, mammalian milk oligosaccharides, chitin, chitosan, casein, yeast, yeast extract, single cell protein, mycoproteins, or other disaccharides or polysaccharides, or mixtures thereof.

15: Lactobacillus cells dried in the process according to claim 1.
